(19) United States
(12) Patent Application Publication
(10) Pub. No.: US 2026/0108717 A1
(43) Pub. Date: Apr. 23, 2026

(54) CONNECTION RETENTION ASSEMBLY

(71) Applicant: CareFusion 303, Inc., San Diego, CA (US)

(72) Inventor: Raymond P. FEITH, Chino Hills, CA (US)

(21) Appl. No.: 18/920,685

(22) Filed: Oct. 18, 2024

(51) Int. Cl. A61M 39/10; A61M 5/14; A61M 25/00

(52) U.S. Cl. CPC A61M 39/1011; A61M 5/1413; A61M 25/0097; A61M 2207/00

(57) ABSTRACT

A connector assembly can serve to couple first and second connectors using a clamp component. The first connector can have a central bore, an outer surface, and a slot extending radially inwardly from the outer surface to the bore. The slot can be configured to receive at least a portion of the clip component therein for restricting longitudinal movement of the clip relative to the first component. The second component can have a mating groove configured to engage with the engagement member of the clip component when the second component is positioned in the central bore of the first component for securing the second component in an engaged configuration relative to the first component. The second component can be removable from the central bore upon exertion of a separating force that dislodges the engagement member from the mating groove of the second component.

### TECHNICAL FIELD

The present disclosure relates generally to medical fluid connectors and, more particularly, to a connector assembly that includes medical connectors that decouple with each other due to an applied force. The decoupling may be due to intentional or unintentional separation between the medical connectors.

### BACKGROUND

Peripheral intravenous (“PIVC”) catheters are medical tools inserted into peripheral veins of patients to deliver medical fluid to the patients. In an example application, the medical fluid is delivered to the patient, and a medical professional subsequently removes the PIVC catheter from the patient. Often, however, these catheters are unintentionally dislodged. For example, catheter lines receiving an unintended or unexpected pulling force can pull the IV tubing, which pulls the catheter out of the patient. In other instances, catheters are accidentally removed from patients and medical professionals. Unintended or unexpected dislodgement can lead to patient blood loss, IV fluid loss, and IV fluid delivery delay.

### SUMMARY

In accordance with at least some embodiments disclosed herein is the realization that unintended dislodgement or disconnection of a medical connection, such as a medical fluid line, can result in injury to a patient or a medical professional, such as by depriving the patient of a medicament, increasing the potential for infection to the patient, and exposing the medical professional to medicaments.

In accordance with some embodiments, the connector assembly can include first and second tubular components, connectors, or shafts, which may be configured to interconnect through inserting, longitudinally overlapping or abutting, or otherwise forming a seal between the first and second tubular components. The connector assembly can also include a clamp component or clip that can engage with at least a portion of the first tubular component or outer shaft and at least a portion of the second tubular component or inner shaft to limit relative motion of the first and second tubular components.

In accordance with some embodiments, the clamp component may serve to prevent or restrict relative axial or longitudinal movement between the first and second tubular components. Optionally, the clamp component may also serve to prevent or restrict relative rotation between the first and second tubular components. Moreover, in accordance with some embodiments, the clamp component may serve to prevent or restrict relative longitudinal and rotational movement between the first and second tubular components.

However, in some embodiments, the clamp component may also advantageously prevent or restrict relative longitudinal movement between the first and second tubular components while permitting relative rotational movement. Such an ability for the first and second tubular components to rotate freely can avoid or eliminate line kinking.

The connector assembly can be configured such that the clamp component is capable of preventing or restricting relative movement between the first and second tubular components below a certain threshold disconnection force between the first and second tubular components.

In some embodiments, the connector assembly can experience a disconnection force (i.e., opposing axial forces pulling the first and second tubular components apart) on the first and second tubular components that may otherwise cause the first and second tubular components to disengage from each other. This disconnection force may cause the first and second tubular components to break or otherwise malfunction, thus damaging and rendering the connector assembly in operable.

The present disclosure provides devices and methods for avoiding such unfortunate outcomes. These devices and methods can ensure patient safety in the event of a disconnection force through the use of a unique clamp-connector assembly concept that enables the connector assembly to maintain engagement between first and second tubular components during normal or acceptable ranges of disconnection force, while permitting the first and second tubular components to become disengage from each other upon application of a sufficient or significant disconnection force.

### BRIEF DESCRIPTION OF THE DRAWINGS

Various features of illustrative embodiments of the inventions are described below with reference to the drawings. The illustrated embodiments are intended to illustrate, but not to limit, the inventions. The drawings contain the following figures:

FIG. 1 illustrates an IV set coupled to a patient, in accordance with aspects of the present disclosure

FIG. 2 illustrates a connector assembly in a disconnected state, in accordance with aspects of the present disclosure.

FIGS. 3A-3C illustrate separate components of the connector assembly of FIG. 2, in accordance with aspects of the present disclosure.

FIGS. 4A and 4B illustrate partial cross-sectional views of the connector assembly in engaged and disengaged configurations, in accordance with aspects of the present disclosure.

FIG. 5 illustrates a partial cross-sectional view of the connector assembly in the engaged configuration, taken along a longitudinal axis of the assembly, in accordance with aspects of the present disclosure.

### DETAILED DESCRIPTION

In the following detailed description, numerous specific details are set forth to provide a full understanding of the subject technology. It should be understood that the subject technology may be practiced without some of these specific details. In other instances, well-known structures and techniques have not been shown in detail so as not to obscure the subject technology.

Further, while the present description sets forth specific details of various embodiments, it will be appreciated that the description is illustrative only and should not be construed in any way as limiting. Additionally, it is contemplated that although particular embodiments of the present disclosure may be disclosed or shown in the context of an IV set, such embodiments can be used in other fluid conveyance systems. Furthermore, various applications of such embodiments and modifications thereto, which may occur to those who are skilled in the art, are also encompassed by the general concepts described herein.

In accordance with some embodiments, the present disclosure provides a connector assembly that has numerous features and advantages for medical applications. The connectors or shafts may each include a means for engaging with an engagement component that can limit, restrict, or prevent rotational and/or longitudinal relative movement between the connectors. This can beneficially allow for safe and easy interconnection while permitting a desired threshold disengagement force to be exerted, whether intentionally or not, to ensure that the assembly is both easy to use and helps to avoid trauma in the event of accidental disconnection.

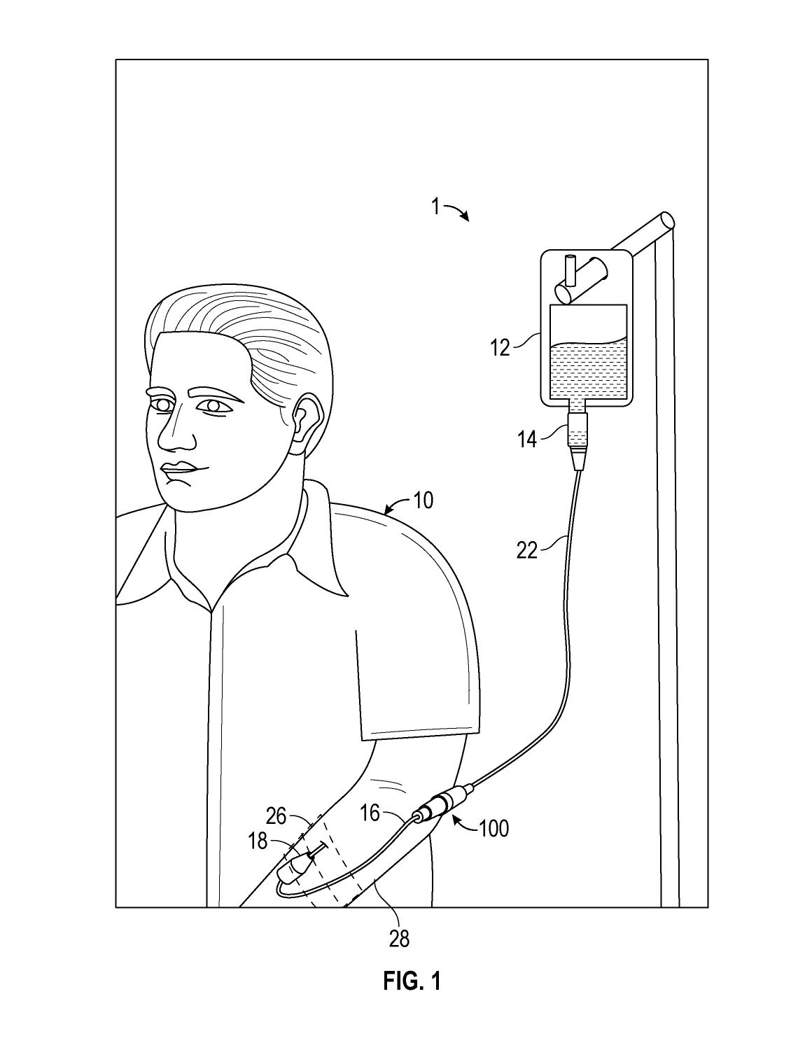

Referring now to the figures, FIG. 1 illustrates an IV set 1 coupled to a patient 10, in accordance with aspects of the present disclosure. The IV set 1 includes a medicament bag 12, a drip chamber 14, and tubing 22. The tubing 22 extends between the drip chamber 14 and a fluid connector assembly 100 of the IV set 1. To resist unintended dislodgement or disconnection of the tubing 16 or the catheter 18 from the patient, tape 26 is placed over the tubing 16 and the catheter 18, so that the tape 26 engages the tubing 16, the catheter 18, and the patient 10.

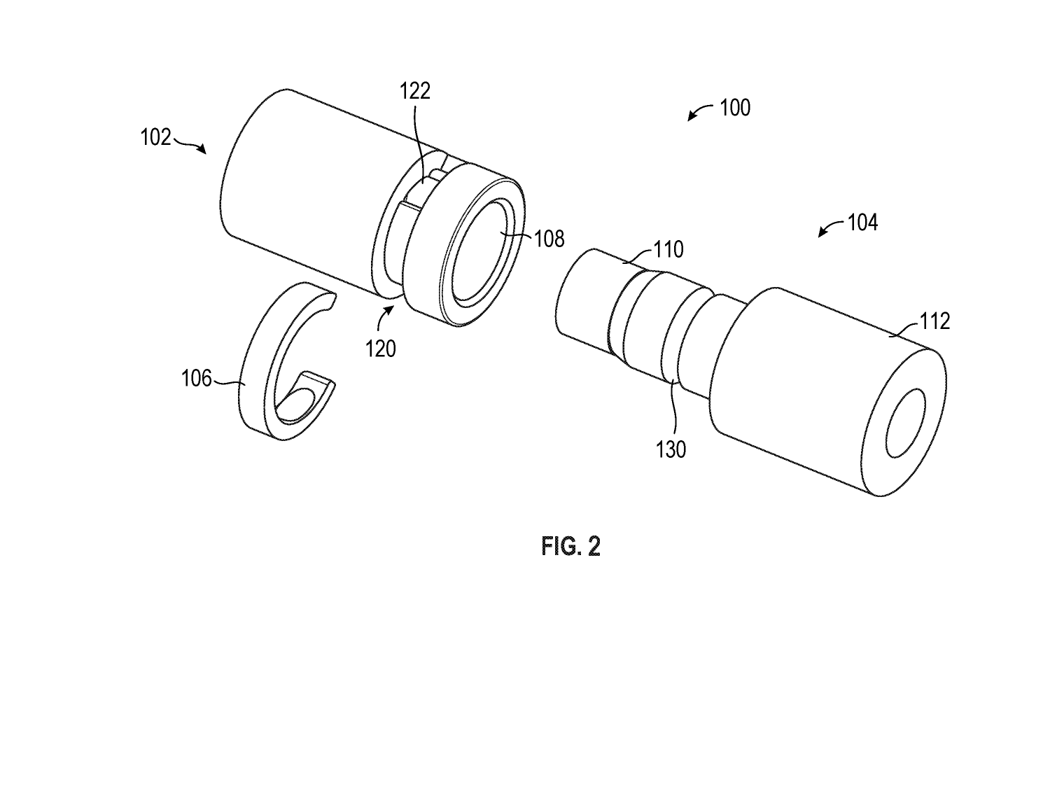

FIG. 2 illustrates a connector assembly 100 in a disconnected state, in accordance with aspects of the present disclosure. In FIG. 2, the connector assembly 100 is shown in a disengaged state. As may be appreciated by a person of skill in the art, the components of the connector can be coupled to respective elements that provide fluid management, such as valves, catheters, and other equipment. The connector assembly 100 is designed for use in medical applications, such as the IV set 1 (shown in FIG. 1) as well as other IV medical fluid delivery applications using catheters, including PIVC catheters, as non-limiting examples.

The connector assembly can include first and second tubular components, which may be configured to interconnect through inserting, longitudinally overlapping or abutting, or otherwise forming a seal between the first and second tubular components, connectors, or shafts. The connector assembly can also include a clamp component that can engage with at least a portion of the first tubular component and at least a portion of the second tubular component to limit relative motion of the first and second tubular components.

In accordance with some embodiments, the clamp component may serve to prevent or restrict relative axial or longitudinal movement between the first and second tubular components. Optionally, the clamp component may also serve to prevent or restrict relative rotation between the first and second tubular components. Moreover, in accordance with some embodiments, the clamp component may serve to prevent or restrict relative longitudinal and rotational movement between the first and second tubular components.

However, in some embodiments, the clamp component may also advantageously prevent or restrict relative longitudinal movement between the first and second tubular components while permitting relative rotational movement. Such an ability for the first and second tubular components to rotate freely can avoid or eliminate line kinking.

The connector assembly can be configured such that the clamp component is capable of preventing or restricting relative movement between the first and second tubular components below a certain threshold disconnection force between the first and second tubular components.

For example, in use situations, the connector assembly will commonly experience opposing forces on the first and second tubular components that may otherwise cause the first and second tubular components to disengage from each other. This disconnection force, if great enough, may cause the first and second tubular components to break or otherwise malfunction, thus damaging and rendering the connector assembly in operable. In order to avoid such unfortunate outcomes, and to ensure patient safety in the event of a significant disconnection force, the present disclosure provides for a unique clamp component concept that enables the connector assembly to maintain engagement between the first and second tubular components during normal or acceptable ranges of disconnection force, while permitting the first and second tubular components to become disengage from each other upon application of a sufficient or significant disconnection force.

In accordance with some embodiments, the connector assembly can be configured to retain the first and second tubular components in an engaged configuration if the disconnection force is less than about 4 pounds, acting in an axial direction. However, if the disconnection force exceeds 4 pounds, the engagement force created by the clamp component and the connector assembly 100 can be overcome, thus permitting disengagement between the first and second tubular components.

The connector assembly can therefore be configured to withstand disconnection forces of up to 2 pounds, up to 2.5 pounds, up to 3 pounds, up to 3.5 pounds, or up to 4 pounds or more, acting in an axial direction on the first and second tubular components. Further, the connector assembly can disengage or allow the first and second tubular components to become separated with a force greater than 3.8 pounds, greater than 3.9 pounds, greater than 4 pounds, greater than 4.1 pounds, greater than 4.2 pounds, greater than 4.3 pounds, greater than 4.4 pounds, or greater than 4.5 pounds. These ranges have been found to provide important benefits for maintaining engagement between the first and second tubular components while permitting disengagement when the disconnection force exceeds such thresholds, thus avoiding patient discomfort and trauma. Furthermore, these ranges also provide remarkably beneficial ease of reconnection and structure preservation (i.e., reducing the chance of breakage or in operability) when attempting to reconnect the first and second tubular components after a disconnection event.

Referring to FIG. 2, the connector assembly 100 can comprise a first connector 102, a second connector 104, and a clamp component 106. The first connector can comprise a receptacle area or a central bore or lumen 108 that is configured to receive at least a portion of the second connector 104 to permit fluid flow between the first connector 102 and the second connector 104.

As shown in the embodiment of the connector assembly 100 of FIG. 2, the second connector 104 can comprise a tubular section 110 that can be inserted into the lumen 108 in order to fluidly connect the first connector 102 and the second connector 104. The tubular section 110 can comprise a reduced diameter relative to a proximal section 112 of the second connector 104 in order to permit the tubular section 110 to be received within the lumen 108.

The first connector 102 can comprise a bay or slot 120 into which the clamp component 106 can at least partially extend. In the embodiment shown in FIG. 2, the clamp component 106 can be inserted into the slot 120 and extend at least partially through the slot 120 via an aperture 142 and protrude radially into the lumen 108. In accordance with some embodiments, the slot 102 can comprise one, two, three, or more apertures that permit passage of a portion of the clamp component 106 radially into the lumen 108.

In accordance with some embodiments, the tubular section 110 of the second connector 104 can comprise a detent or groove that can be engaged by the portion of the clamp component 106 that extends into the lumen 108. As shown in the embodiment of FIG. 2, the tubular section 110 can comprise a circumferential groove 130 that has a generally constant depth and shape, extending circumferentially about an outer surface of the tubular section 110.

Although the groove 130 is shown as a continuous, unbroken groove in FIG. 2, some embodiments can be configured to include more than one groove, positioned at distinct longitudinal locations along a length of the tubular section 110. Further, the groove can also be configured to be discontinuous, as a plurality of detents that extend into the surface of the tubular section 110 that different circumferential positions around the tubular section 110.

Optionally, in accordance with some embodiments, the groove can comprise two or more grooves that have different depths in order to permit different degrees of engagement between the clamp component 106 and the groove (e.g., permitting more or less of the clamp component 1062 protrude radially into the groove), thus allowing a clinician to selectively engage a given groove in order to achieve a desired strength of engagement (i.e., enabling the connector assembly to withstand different levels of disconnection forces depending on the groove that is engaged with the clamp component).

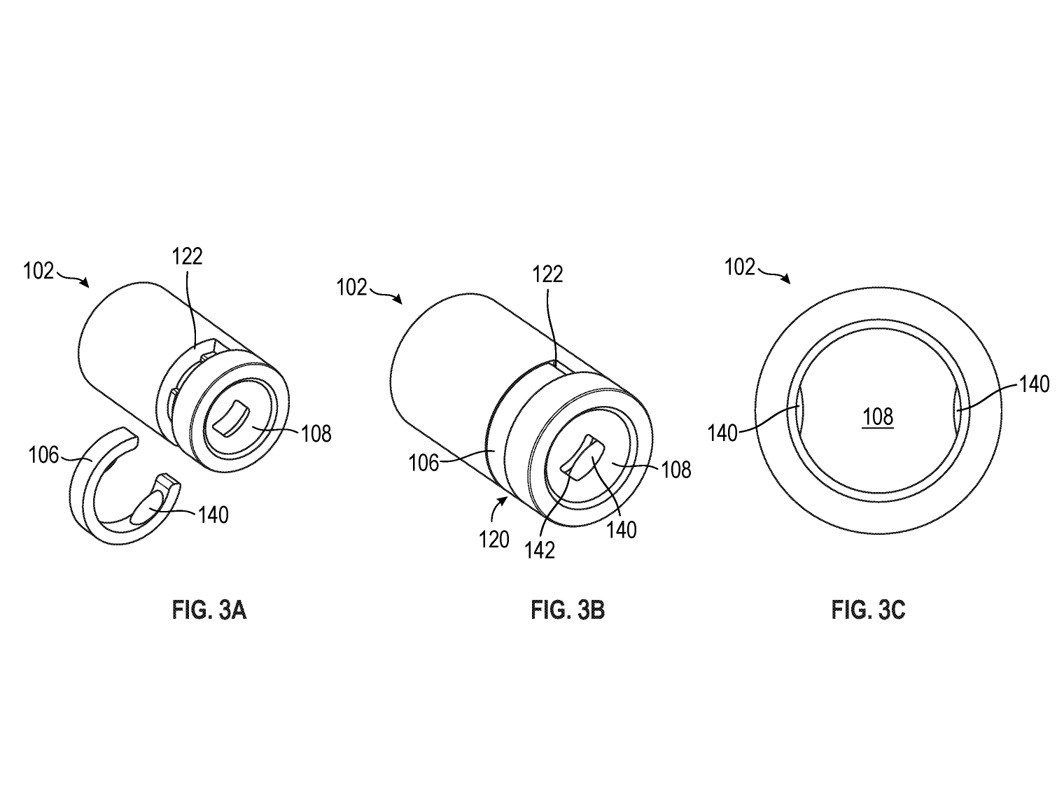

FIGS. 3A-B illustrate the interconnection between the clamp component 106 and the first connector 102. As discussed above, the clamp component 106 can be inserted into the slot 122 of the first connector 102. FIG. 3B illustrates a nested or engaged configuration between the clamp component 106 and the first connector 102. In accordance with some embodiments, an outer surface of the clamp component 106 can advantageously be flush or generally continuous with an outer surface of the first connector 102, thereby avoiding accidental disconnection events or otherwise dislodging the clamp component 106 from the slot 122 of the first connector 102.

Additionally, as illustrated in FIG. 3B, the clamp component 106 can comprise a protruding member 140 that fits into an aperture 142 of the slot 122, thereby permitting the protruding member 140 to protrude radially into the lumen 108, as shown in FIG. 3C.

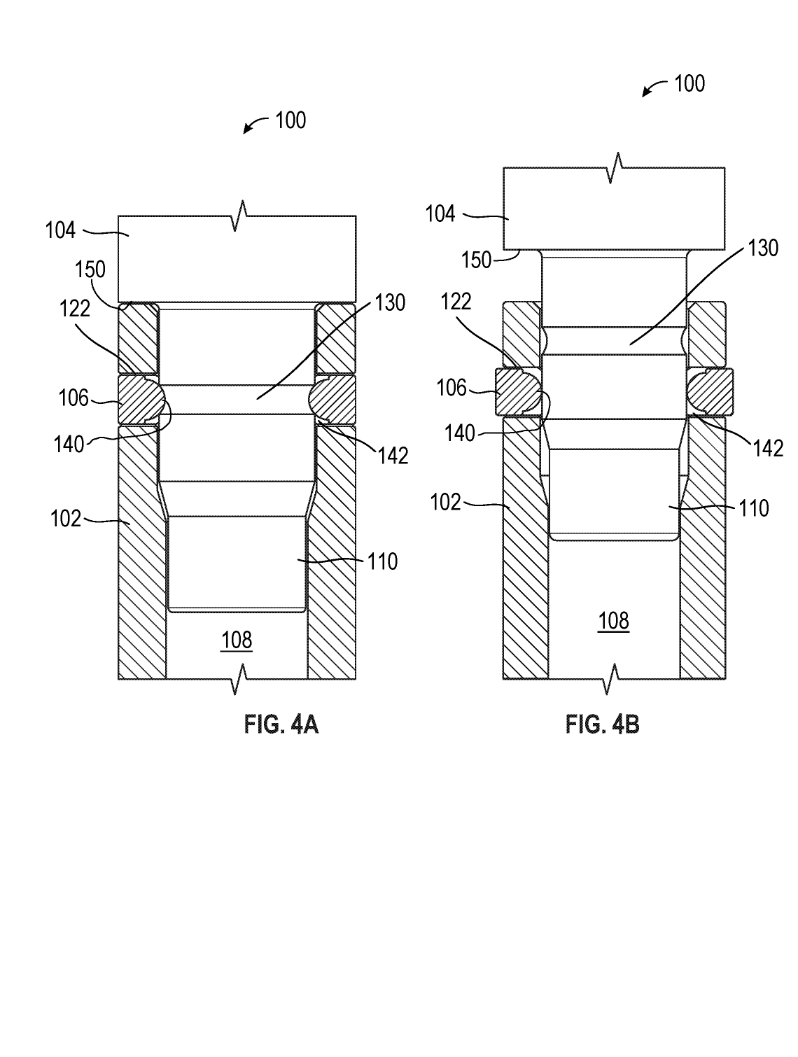

Referring now to FIGS. 4A and 4B, the connector assembly 100 is shown in an engaged configuration (FIG. 4A) and in a disengaged configuration (FIG. 4B).

When in the engaged configuration, the protruding member 140 of the clamp component 106 can extend radially into the groove 130, as shown in FIG. 4A. As discussed above, the degree of engagement between the protruding member 140 and the groove 130 can depend on the structure and number of the protruding member 140 and the groove 130. In the illustrated example, the protruding member 140 can extend radially into the groove 130 and permit the free rotation of the second connector 104 relative to the first connector 102. Such an ability for the first and second connectors 102, 104 to rotate freely can avoid or eliminate line kinking. However, circumferential stops or other structures can be implemented as part of the roof structure in order to limit or restrict relative rotational movement between the first connector 102 and the second connector 104.

Additionally, as shown in FIG. 4A the lumen 108 of the first connector 102 can be configured to mate with the outer surface of the tubular section 110. For example, as shown, the lumen 108 can have a stepped inner profile, through which the inner diameter of the lumen 108 changes or tapers to a smaller diameter in a direction away from an entrance or proximal end of the lumen 108 of the first connector 102. The tubular section 110 can comprise a stepped outer profile that can match the stepped inner profile of the lumen 108. As illustrated in FIG. 4A, in the engaged position, the clamp component 106 is engaged with the groove 130 and the longitudinal alignment of the first connector 102 relative to the second component 104 is such that the stepped outer profile of the tubular section 110 can mate against, contact, or be positioned in close alignment with the stepped inner profile of the lumen 108. In this manner, the engaged configuration can be achieved when as the second connector 104 is inserted at precisely the correct depth into the lumen 108 of the first connector 102 so that the groove 130 is aligned with the protruding member 140 and a shoulder 150 of the second connector 104 abuts a proximal end portion of the first connector 102.

Referring to FIG. 4B, upon exertion of a sufficient disconnection force, as discussed above, the second connector 104 can be longitudinally removed from the lumen 108 of the first connector 102. During this process, the protruding member 140 can be forced out of the groove 130 due to the disconnection force.

In some embodiments, the groove 130 and the protruding member 140 can have corresponding geometries that permit a desired engagement. These features can define surfaces that can slide relative to each other during the disengagement process as the axial disconnection force overcomes the radial force of the clamp component 106. In other words, the clamp component 106 will tend to resist radial expansion, thus urging the protruding members 140 radially inward once the clamp component 106 is positioned within the slot 122.

For example, in some embodiments, the groove 130 and the protruding member 140 can each comprise rounded surfaces that can mate against each other. However, it is contemplated that other geometries, such as squared, triangular, or other such cross-sections may be implemented in order to initial resistance after which failure of the relative engagement will permit separation of the first and second connectors 102, 104.

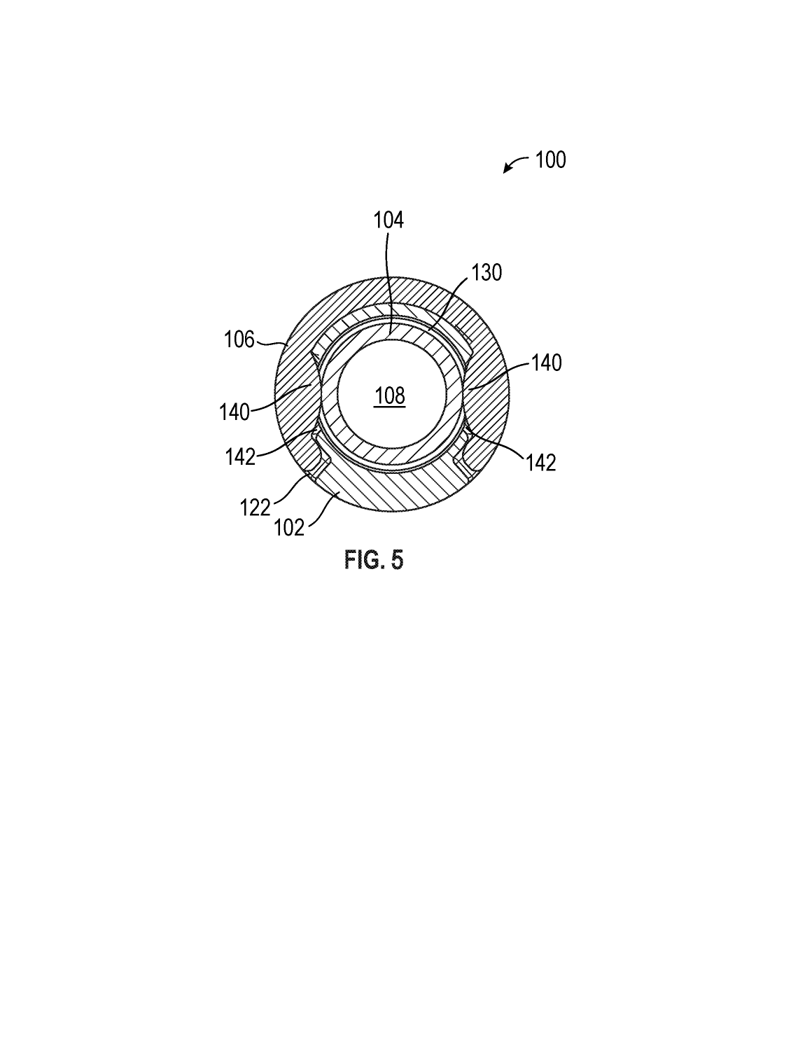

FIG. 5 illustrates an end, cross-sectional view of the connector assembly taken along the longitudinal axis of the connector assembly. As discussed above, the protruding members 140 can extend through the apertures 142 of the first connector 102 in order to engage with a groove 130 of the first connector 102. As shown, the clamp component 106 can comprise to protruding members.

Further, in accordance with some embodiments, the clamp component 106 and the slot 122 can extend circumferentially about at least half of the circumference of the outer and inner shaft of the first and second connectors 102, 104. The degree of the circumferential overlap or extension of these components can be at least half, 60%, 70%, or more of the circumference of these components.

As illustrated and discussed above, the connector assembly 100 can be operated using a simple procedure of placing the first and second connectors 102, 104 into a fluidic coupling configuration, such as by inserting the second connector 104 into the first connector 102. The clamp component 106 can engage with the second connector 104 when both are coupled to or positioned in the engaged configuration with the first connector 102. The clamp component 106 can be coupled to the first connector 102 either before or after the second connectors 104 is inserted into or coupled to the first connector 102. Thereafter, if dislodged or disconnected, the second connector 104 can be reattached or reengaged with the first connector 102 by the clinician with facility.

The features of the present disclosure provide first and second compressible members that can be used as valves to regulate a fluid pathway therebetween. The first and second compressible members are located in a first and a second connector, respectively. If the first and second connectors are separated, whether unintentionally or intentionally, the fluid pathway for each of the first and second compressible members become closed or obstructed to prevent fluid loss therefrom. The features of the present disclosure also provide that upon separation of the first and second compressible members, any of the first and second compressible members can be cleaned and disinfected, and the first and second compressible members can be once again coupled together to form a fluid pathway therebetween.

### Illustration of Subject Technology as Clauses

The subject technology is illustrated, for example, according to various aspects described below. Various examples of aspects of the subject technology are described as numbered clauses (1, 2, 3, etc.) for convenience. These are provided as examples and do not limit the subject technology. It is noted that any of the dependent clauses may be combined in any combination, and placed into a respective independent clause, e.g., clause 1, clause 11, clause 17, clause, clause 23, clause 26, or clause 29. The other clauses can be presented in a similar manner.

Clause 1. A connector assembly, comprising: a clip component having an engagement member; an outer shaft having a central bore, an outer surface, and a slot extending radially inwardly from the outer surface to the bore, the slot being configured to receive at least a portion of the clip component therein for restricting longitudinal movement of the clip relative to the outer shaft; and an inner shaft having a mating groove configured to engage with the engagement member of the clip component when the inner shaft is positioned in the central bore of the outer shaft for securing the inner shaft in an engaged configuration relative to the outer shaft, the inner shaft being removable from the central bore upon exertion of a separating force that dislodges the engagement member from the mating groove of the inner shaft.

Clause 2. The connector assembly of clause 1, wherein the clip comprises a circular ring.

Clause 3. The connector assembly of any of the preceding clauses, wherein the clip comprises a C-shape.

Clause 4. The connector assembly of any of the preceding clauses, wherein the clip fits entirely within the slot in a coupled configuration.

Clause 5. The connector assembly of any of the preceding clauses, wherein an outer surface of the clip is contiguous with the outer surface of the outer shaft.

Clause 6. The connector assembly of any of the preceding clauses, wherein the engagement member of the clip comprises a radial protrusion that extends radially inwardly into the mating area of the inner shaft when in the assembled condition.

Clause 7. The connector assembly of any of the preceding clauses, wherein the inner shaft comprises first and second sections, the first section having a diameter that is smaller than a diameter of the second section.

Clause 8. The connector assembly of clause 7, wherein the first section comprises the mating groove.

Clause 9. The connector assembly of clause 7, wherein the inner shaft comprises a cylindrical profile.

Clause 10. The connector assembly of any of the preceding clauses, wherein the slot extends circumferentially about at least 50% of the circumference of the outer shaft.

Clause 11. The connector assembly of any of the preceding clauses, wherein the slot extends circumferentially about at least 60% of the circumference of the outer shaft.

Clause 12. The connector assembly of any of the preceding clauses, wherein the slot extends circumferentially about at least 70% of the circumference of the outer shaft.

Clause 13. The connector assembly of any of the preceding clauses, wherein the slot extends circumferentially about at least 75% of the circumference of the outer shaft.

Clause 14. The connector assembly of any of the preceding clauses, wherein the mating groove extends circumferentially about at least half of the circumference of the inner shaft.

Clause 15. The connector assembly of any of the preceding clauses, wherein the mating groove extends circumferentially about at least 60% of the circumference of the inner shaft.

Clause 16. The connector assembly of any of the preceding clauses, wherein the mating groove extends circumferentially about at least 70% of the circumference of the inner shaft.

Clause 17. A connector assembly, comprising: an outer shaft having a central bore and a slot that extends from an outer surface thereof radially inwardly to the central bore; an inner shaft that can fit into the outer shaft bore, the inner shaft having a mating groove that can be aligned with the slot when the inner shaft is inserted into the outer shaft bore; and a clamp that can be secured onto the outer shaft and extend at least partially through the slot into the mating groove for restricting at least one of rotation or axial movement of the outer shaft relative to the inner shaft.

Clause 18. The connector assembly of clause 17, wherein the clamp comprises a circular ring.

Clause 19. The connector assembly of any of clauses 17–18, wherein the clamp comprises a C-shape.

Clause 20. The connector assembly of any of clauses 17–19, wherein an outer surface of the clamp is contiguous with an outer surface of the outer shaft.

Clause 21. The connector assembly of any of clauses 17–20, wherein the clamp comprises an engagement member having a radial protrusion that extends radially inwardly into the mating groove of the inner shaft when in an assembled condition.

Clause 22. The connector assembly of any of clauses 17–21, wherein the inner shaft comprises first and second sections, the first section having a diameter that is smaller than a diameter of the second section.

Clause 23. The connector assembly of clause 22, wherein the first section comprises the mating groove.

Clause 24. The connector assembly of clause 22, wherein the inner shaft comprises a cylindrical profile.

Clause 25. The connector assembly of any of clauses 17–24, wherein the slot extends circumferentially about at least 50% of the circumference of the outer shaft.

Clause 26. The connector assembly of any of clauses 17–25, wherein the slot extends circumferentially about at least 60% of the circumference of the outer shaft.

Clause 27. The connector assembly of any of clauses 17–26, wherein the slot extends circumferentially about at least 70% of the circumference of the outer shaft.

Clause 28. The connector assembly of any of clauses 17–27, wherein the slot extends circumferentially about at least 75% of the circumference of the outer shaft.

Clause 29. The connector assembly of any of clauses 17–28, wherein the mating groove extends circumferentially about at least half of the circumference of the inner shaft.

Clause 30. The connector assembly of any of clauses 17–29, wherein the mating groove extends circumferentially about at least 60% of the circumference of the inner shaft.

Clause 31. The connector assembly of any of clauses 17–30, wherein the mating groove extends circumferentially about at least 70% of the circumference of the inner shaft.

Clause 32. The connector assembly of any of clauses 17–31, wherein the inner shaft is removable from the central bore upon exertion of a separating force that dislodges the clamp from the mating groove of the inner shaft.

Clause 33. A method of securing a connector assembly, comprising: inserting an inner shaft into a central bore of an outer shaft, the inner shaft having a mating groove extending about at least a portion of the inner shaft, the outer shaft having a slot that extends from an outer surface thereof radially inwardly to the central bore; coupling a clamp with the outer shaft; and engaging a protruding member of the clamp with the mating groove in order to restrict at least one of rotation or axial movement of the outer shaft relative to the inner shaft.

Clause 34. The method of clause 33, further comprising aligning the mating groove of the inner shaft with the slot of the outer shaft.

Clause 35. The method of any of clauses 33–34, wherein the engaging comprises inserting the engagement member onto the outer shaft and extend at least a portion of the engagement member through the slot into the mating groove.

Clause 36. The method of any of clauses 33–35, wherein the coupling comprises longitudinally aligning the clamp with the slot.

Clause 37. The method of any of clauses 33–36, wherein the coupling comprises positioning an outer surface of the clamp contiguous with an outer surface of the outer shaft.

Clause 38. The method of any of clauses 33–37, wherein the engaging comprises restricting longitudinal movement of the inner shaft relative to the outer shaft.

Clause 39. The method of clause 38, where the engaging comprises permitting rotational movement of the inner shaft relative to the outer shaft.

Clause 40. The assembly or methods of any of the preceding clauses, wherein the assembly comprises a valve retention ring assembly.

### Further Considerations

In some embodiments, any of the clauses herein may depend from any one of the independent clauses or any one of the dependent clauses. In one aspect, any of the clauses (e.g., dependent or independent clauses) may be combined with any other one or more clauses (e.g., dependent or independent clauses). In one aspect, a claim may include some or all of the words (e.g., steps, operations, means or components) recited in a clause, a sentence, a phrase or a paragraph. In one aspect, a claim may include some or all of the words recited in one or more clauses, sentences, phrases or paragraphs. In one aspect, some of the words in each of the clauses, sentences, phrases or paragraphs may be removed. In one aspect, additional words or elements may be added to a clause, a sentence, a phrase or a paragraph. In one aspect, the subject technology may be implemented without utilizing some of the components, elements, functions or operations described herein. In one aspect, the subject technology may be implemented utilizing additional components, elements, functions or operations.

The present disclosure is provided to enable any person skilled in the art to practice the various aspects described herein. The disclosure provides various examples of the subject technology, and the subject technology is not limited to these examples. Various modifications to these aspects will be readily apparent to those skilled in the art, and the generic principles defined herein may be applied to other aspects.

A reference to an element in the singular is not intended to mean “one and only one” unless specifically so stated, but rather “one or more.” Unless specifically stated otherwise, the term “some” refers to one or more. Pronouns in the masculine (e.g., his) include the feminine and neuter gender (e.g., her and its) and vice versa. Headings and subheadings, if any, are used for convenience only and do not limit the invention.

The word “exemplary” is used herein to mean “serving as an example or illustration.” Any aspect or design described herein as “exemplary” is not necessarily to be construed as preferred or advantageous over other aspects or designs. In one aspect, various alternative configurations and operations described herein may be considered to be at least equivalent.

A phrase such as an “aspect” does not imply that such aspect is essential to the subject technology or that such aspect applies to all configurations of the subject technology. A disclosure relating to an aspect may apply to all configurations, or one or more configurations. An aspect may provide one or more examples. A phrase such as an aspect may refer to one or more aspects and vice versa. A phrase such as an “embodiment” does not imply that such embodiment is essential to the subject technology or that such embodiment applies to all configurations of the subject technology. A disclosure relating to an embodiment may apply to all embodiments, or one or more embodiments. An embodiment may provide one or more examples. A phrase such an embodiment may refer to one or more embodiments and vice versa. A phrase such as a “configuration” does not imply that such configuration is essential to the subject technology or that such configuration applies to all configurations of the subject technology. A disclosure relating to a configuration may apply to all configurations, or one or more configurations. A configuration may provide one or more examples. A phrase such a configuration may refer to one or more configurations and vice versa.

In one aspect, unless otherwise stated, all measurements, values, ratings, positions, magnitudes, sizes, and other specifications that are set forth in this specification, including in the claims that follow, are approximate, not exact. In one aspect, they are intended to have a reasonable range that is consistent with the functions to which they relate and with what is customary in the art to which they pertain.

In one aspect, the term “coupled” or the like may refer to being directly coupled. In another aspect, the term “coupled” or the like may refer to being indirectly coupled.

Terms such as “top,” “bottom,” “front,” “rear” and the like if used in this disclosure should be understood as referring to an arbitrary frame of reference, rather than to the ordinary gravitational frame of reference. Thus, a top surface, a bottom surface, a front surface, and a rear surface may extend upwardly, downwardly, diagonally, or horizontally in a gravitational frame of reference.

Various items may be arranged differently (e.g., arranged in a different order, or partitioned in a different way) all without departing from the scope of the subject technology. All structural and functional equivalents to the elements of the various aspects described throughout this disclosure that are known or later come to be known to those of ordinary skill in the art are expressly incorporated herein by reference and are intended to be encompassed by the claims. Moreover, nothing disclosed herein is intended to be dedicated to the public regardless of whether such disclosure is explicitly recited in the claims. No claim element is to be construed under the provisions of 35 U.S.C. §112, sixth paragraph, unless the element is expressly recited using the phrase “means for” or, in the case of a method claim, the element is recited using the phrase “step for.” Furthermore, to the extent that the term “include,” “have,” or the like is used, such term is intended to be inclusive in a manner similar to the term “comprise” as “comprise” is interpreted when employed as a transitional word in a claim.

The Title, Background, Summary, Brief Description of the Drawings and Abstract of the disclosure are hereby incorporated into the disclosure and are provided as illustrative examples of the disclosure, not as restrictive descriptions. It is submitted with the understanding that they will not be used to limit the scope or meaning of the claims. In addition, in the Detailed Description, it can be seen that the description provides illustrative examples and the various features are grouped together in various embodiments for the purpose of streamlining the disclosure. This method of disclosure is not to be interpreted as reflecting an intention that the claimed subject matter requires more features than are expressly recited in each claim. Rather, as the following claims reflect, inventive subject matter lies in less than all features of a single disclosed configuration or operation. The following claims are hereby incorporated into the Detailed Description, with each claim standing on its own as a separately claimed subject matter.

The claims are not intended to be limited to the aspects described herein but is to be accorded the full scope consistent with the language claims and to encompass all legal equivalents. Notwithstanding, none of the claims are intended to embrace subject matter that fails to satisfy the requirement of 35 U.S.C. §101, 102, or 103, nor should they be interpreted in such a way.

What is claimed is:

1. A connector assembly, comprising:
a clip component having an engagement member;
an outer shaft having a central bore, an outer surface, and a slot extending radially inwardly from the outer surface to the bore, the slot being configured to receive at least a portion of the clip component therein for restricting longitudinal movement of the clip relative to the outer shaft; and
an inner shaft having a mating groove configured to engage with the engagement member of the clip component when the inner shaft is positioned in the central bore of the outer shaft for securing the inner shaft in an engaged configuration relative to the outer shaft, the inner shaft being removable from the central bore upon exertion of a separating force that dislodges the engagement member from the mating groove of the inner shaft.

2. The connector assembly of claim 1, wherein the clip comprises a circular ring.

3. The connector assembly of claim 1, wherein the clip comprises a C-shape.

4. The connector assembly of claim 1, wherein the clip fits entirely within the slot in a coupled configuration.

5. The connector assembly of claim 1, wherein the engagement member of the clip comprises a radial protrusion that extends radially inwardly into the mating groove of the inner shaft when in the assembled condition.

6. The connector assembly of claim 1, wherein the inner shaft comprises first and second sections, the first section having a diameter that is smaller than a diameter of the second section.

7. The connector assembly of claim 6, wherein the first section comprises the mating groove.

8. The connector assembly of claim 1, wherein the slot extends circumferentially about at least 50% of the circumference of the outer shaft.

9. The connector assembly of claim 1, wherein the mating groove extends circumferentially about at least half of the circumference of the inner shaft.

10. A connector assembly, comprising:
an outer shaft having a central bore and a slot that extends from an outer surface thereof radially inwardly to the central bore;
an inner shaft that can fit into the outer shaft bore, the inner shaft having a mating groove that can be aligned with the slot when the inner shaft is inserted into the outer shaft bore; and
a clamp that can be secured onto the outer shaft and extend at least partially through the slot into the mating groove for restricting at least one of rotation or axial movement of the outer shaft relative to the inner shaft.

11. The connector assembly of claim 10, wherein the clamp comprises an engagement member having a radial protrusion that extends radially inwardly into the mating groove of the inner shaft when in an assembled condition.

12. The connector assembly of claim 10, wherein the inner shaft comprises first and second sections, the first section having a diameter that is smaller than a diameter of the second section.

13. The connector assembly of claim 12, wherein the first section comprises the mating groove.

14. The connector assembly of claim 10, wherein the slot extends circumferentially about at least 50% of the circumference of the outer shaft.

15. The connector assembly of claim 10, wherein the mating groove extends circumferentially about at least half of the circumference of the inner shaft.

16. A method of securing a connector assembly, comprising:
inserting an inner shaft into a central bore of an outer shaft, the inner shaft having a mating groove extending about at least a portion of the inner shaft, the outer shaft having a slot that extends from an outer surface thereof radially inwardly to the central bore;
coupling a clamp with the outer shaft; and
engaging a protruding member of the clamp with the mating groove in order to restrict at least one of rotation or axial movement of the outer shaft relative to the inner shaft.

17. The method of claim 16, further comprising aligning the mating groove of the inner shaft with the slot of the outer shaft.

18. The method of claim 16, wherein the engaging comprises inserting the engagement member onto the outer shaft and extend at least a portion of the engagement member through the slot into the mating groove.

19. The method of claim 16, wherein the engaging comprises restricting longitudinal movement of the inner shaft relative to the outer shaft.

20. The method of claim 19, where the engaging comprises permitting rotational movement of the inner shaft relative to the outer shaft.
